SEQUENCE LISTING (14 sequences)

SEQ ID NO: 1 - DNA, 4 bp, Homo sapiens
tcta

SEQ ID NO: 2 - DNA, 4 bp, Homo sapiens
agat

SEQ ID NO: 3 - DNA, 4 bp, Homo sapiens
agaa

SEQ ID NO: 4 - DNA, 4 bp, Homo sapiens
aaag

SEQ ID NO: 5 - DNA, 4 bp, Homo sapiens
aatg

SEQ ID NO: 6 - DNA, 4 bp, Homo sapiens
tttc

SEQ ID NO: 7 - DNA, 4 bp, Homo sapiens
cttt

SEQ ID NO: 8 - DNA, 4 bp, Homo sapiens
gata

SEQ ID NO: 9 - DNA, 5 bp, Homo sapiens
aaaga

SEQ ID NO: 10 - DNA, 4 bp, Homo sapiens
tcta

SEQ ID NO: 11 - DNA, 24 bp, Homo sapiens
tctatctgtctgtctatctatcta

SEQ ID NO: 12 - DNA, 4 bp, Homo sapiens
tctg

SEQ ID NO: 13 - DNA, 37 bp, Homo sapiens
tctatctatctatatctatctatctatcatctatcta

SEQ ID NO: 14 - DNA, 51 bp, Homo sapiens
tctatctatctatctatatctatctatctatcatctatctatccatatcta

(19) United States
(12) Patent Application Publication
(10) Pub. No.: US 2005/0112591 A1
(43) Pub. Date: May 26, 2005

(54) Novel method for isolating single stranded product

(75) Inventors: Pero Dimsoski, Belmont, CA (US); Sam Lee Woo, Redwood City, CA (US)

Correspondence Address: MILA KASAN, PATENT DEPT.;APPLIED BIOSYSTEMS, 850 LINCOLN CENTRE DRIVE, FOSTER CITY, CA, 94404, US

(73) Assignee: Applera Corporation, Foster City, CA (US)

(21) Appl. No.: 10/723,388

(22) Filed: Nov. 25, 2003

(51) Int. Cl. C12Q 1/68; C12P 19/34

(52) U.S. Cl. 435/6; 435/91.2

(57) ABSTRACT

The present teachings relate to methods of purifying, isolating, separating, and identifying target nucleic acids. In some embodiments of the present teachings, an affinity moiety can be incorporated into one of the flanking primers of a nucleic acid amplification reaction primer pair. The reaction mixture can be contacted with a binding moiety specific for the affinity moiety, thereby allowing immobilization of the double stranded amplification product, separation of reaction components lacking the affinity moiety, and isolation of the target nucleic acid strand. Further, one of the flanking primers of the nucleic acid amplification reaction can comprise a label and a mobility modifier, thereby facilitating identification of the target nucleic acids. In some embodiments, the amplification reaction is multiplexed and comprises polymorphic microsatellites useful in human identification. and the manufacturing of molecular size standards. Some embodiments of the present teachings provide for improved methods of performing electrokinetic injection.

### FIELD

The present teachings relate to methods for separating, isolating, and purifying nucleic acids in the field of molecular biology.

### BACKGROUND REFERENCES

Ausubel et al., CURRENT PROTOCOLS IN MOLECULAR BIOLOGY, John Wiley & Sons, New York, 2001
THE POLYMERASE CHAIN REACTION, Mullis, K. B., F. Ferre, and R. A. Gibbs, Eds., MOLECULAR CLONING: A LABORATORY MANUAL (3rd ed.) Sambrook, J. & D. Russell, =Eds. Cold Spring Harbor Laboratory Press, Cold Spring Harbor, N.Y. (2001).
U.S. application Ser. No. 09/850,514
U.S. application Ser. No. 09/850,590
U.S. application Ser. No. 09/998,887
U.S. Pat. No. 6,124,092
U.S. Pat. No. 6,207,818
Published U.S. application Ser. No. 09/908,994
Belgrader et al., 1996
Ruiz-Martinez et al., 1998
Salas-Solano et al., 1998
U.S. Pat. No. 5,470,705
U.S. Pat. No. 5,514,543
U.S. Pat. No. 5,580,732
U.S. Pat. No. 5,624,800
U.S. Pat. No. 5,807,682
PCT Publication No. WO 01/92579
Wenz, H. et al. (1998) Genome Res. 8:69-80
Christensen, M. et al. (1999) Scand. J Clin. Lab. Invest. 59(3):167-177.
Butler et al., 2003
Grubwieser et al., 2003
Wiegand et al., 2001
Tsukada et al., 2002
Hellmann et al., 2001
Matthews et al. (1988)
Haugland (1992)
Keller and Manak (1993)
Eckstein (1991)
Kricka (1992)
Fre'geau et al. (1993) Biotechniques 15:100-119
G. T. Hermanson, Bioconjugate Techniques, Academic Press, San Diego, Calif. (1996) S. L. Beaucage et al., Current Protocols in Nucleic Acid Chemistry, John Wiley & Sons, New York, N.Y. (2000).
Tautz, D. et al. (1986) Nature 322(6080):652-656
U.S. Pat. No. 5,470,705
U.S. Pat. No. 5,514,543
U.S. application Ser. No. 09/836,704
Fields and Noble, Int. J Peptide Protein Res., 35: 161-214 (1990)
Levenson et al., U.S. Pat. No. 4,914,210
http://www.cstl.nist.gov/biotech/strbase/
U.S. Pat. No. 5,874,217
U.S. Pat. No. 6,605,451
Tully et al., Int J Legal Med. 1999;112(4):241-8
Tully et al., Genomics 1996; 34:107-113
Ross et al., Anal. Chem. 1997; 69:3966-3972.
Wojciechowski et al., Clinical Chemistry 45:9:1999.

### INTRODUCTION

Numerous nucleic acid assays in the field of molecular biology involve complex reaction mixtures. Separation, isolation, and purification of components of these reaction mixtures is desirable. The present teachings pertain to separating, isolating, and purifying components from complex reaction mixtures in the field of molecular biology. Some embodiments of the present teachings pertain to analyzing single stranded target polynucleotides following amplification of polymorphic microsatellites, which can be derived from degraded samples.

### SUMMARY

Some embodiments of the present teachings relate to a method for isolating a labeled single stranded target polynucleotide comprising forming a polymerase chain reaction (PCR). The PCR comprises,
a. a polynucleotide region of interest,
b. a first primer specific for the region of interest, wherein the primer has a label and a mobility modifier,
c. a second primer specific for the region of interest, wherein the second primer comprises an affinity moiety, thereby forming a reaction mixture.

The region of interest is amplified, thereby producing a double stranded polynucleotide amplification product. The amplification product comprises the labeled single stranded target polynucleotide comprising the label and the mobility modifier, and a complementary affinity moiety strand. The reaction mixture is contacted with a binding moiety specific for the affinity moiety, thereby binding the double stranded polynucleotide amplification product to the binding moiety. The unbound unincorporated reaction components are removed, and, the labeled single stranded target polynucleotide is released from the bound double stranded polynucleotide amplification product.

In some embodiments, said mobility modifier is chosen from at least one of the group comprising nucleotides, polyethylene oxide, polyglycolic acid, polylactic acid, polypeptide, oligosaccharide, and polyurethane, polyamide, polysulfonamide, polysulfoxide, and block copolymers thereof, including polymers composed of units of multiple subunits linked by charged or uncharged linking groups, and combinations thereof.

In some embodiments, the binding moiety is streptavidin.

In some embodiments, the affinity moiety is biotin.

In some embodiments, the PCR mixture further comprises a plurality of primer pairs, wherein each primer pair comprises a first primer and a second primer that flanks a region of interest, wherein the first primer further comprises the label and the mobility modifier, and wherein the second primer further comprises the affinity moiety.

In some embodiments, the polynucleotide region of interest is derived from a sample that further comprises degraded DNA.

In some embodiments, said degraded DNA is between about 60 and 240 nucleotides in length.

In some embodiments, the regions of interest further comprise polymorphic micro satellites.

In some embodiments, the polymorphic microsatellites further comprise a dinucleotide repeat.

In some embodiments, the polymorphic microsatellites further comprise a trinucleotide repeat.

In some embodiments, the polymorphic microsatellites further comprise a tetranucleotide repeat.

In some embodiments, at least one of the isolated labeled single stranded target polynucleotide results from amplification with a primer pair lacking a mobility modifier.

In some embodiments, the PCR mixture further comprises sorbitol.

In some embodiments, the PCR mixture further comprises betaine.

In some embodiments, the PCR mixture further comprises sorbitol and betaine.

In some embodiments, the present teachings relate to a method for manufacturing a labeled single stranded target polynucleotide molecular size standard comprising forming a PCR mixture. The PCR mixture comprises,
a. a polynucleotide region of interest,
b. a first primer specific for the region of interest, wherein the first primer comprises a label and a mobility modifier, and,
c. a second primer specific for the region of interest, wherein the second primer comprises an affinity moiety.

The region of interest is amplified, thereby producing a double stranded polynucleotide amplification product comprising the single stranded target polynucleotide molecular size standard comprising the label and the mobility modifier, and a complementary affinity moiety strand. The reaction mixture is contacted with a binding moiety specific for the affinity moiety, the double stranded polynucleotide is bound to the binding moiety, the unbound unincorporated reaction components removed, and the labeled single stranded target polynucleotide molecular size standard is released.

Some embodiments of the present teachings further comprise a plurality of regions of interest and a plurality of primer pairs, wherein a plurality of labeled single stranded target polynucleotide molecular size standards is formed.

Some embodiments of the present teachings relate to methods for isolating a labeled single stranded target polynucleotide comprising, forming a PCR mixture comprising,
a. a polynucleotide region of interest,
b. a first primer specific for the region of interest, and,
c. a second primer specific for the region of interest, wherein the second primer comprises an affinity moiety,

The region of interest is amplified, whereby a double stranded polynucleotide amplification product is produced, comprising an unlabelled single strand target polynucleotide, and a complementary affinity moiety strand. The reaction mixture is contacted with a binding moiety specific for the affinity moiety, the double stranded polynucleotide amplification product is bound to the binding moiety, the unbound unincorporated reaction components are removed, and the unlabelled single stranded target polynucleotide is eluted and removed. The following components are then provided,
a. a polymerase,
b. a primer complementary to the bound second strand, wherein the primer further comprises a mobility modifier, and,
c. at least one dye-labelled nucleotide.

An extension reaction is performed to form a labeled single stranded target polynucleotide, and the labeled single stranded target polynucleotide is released.

In some embodiments of the present teachings, the labeled single stranded target polynucleotide is analyzed by a mobility dependent analysis technique.

In some embodiments of the present teachings, the mobility dependent analysis technique further comprises capillary electrophoresis.

### BRIEF DESCRIPTIONS OF THE FIGURES

FIG. 1 shows a schematic of a separation procedure in accordance with some embodiments of the present teachings.

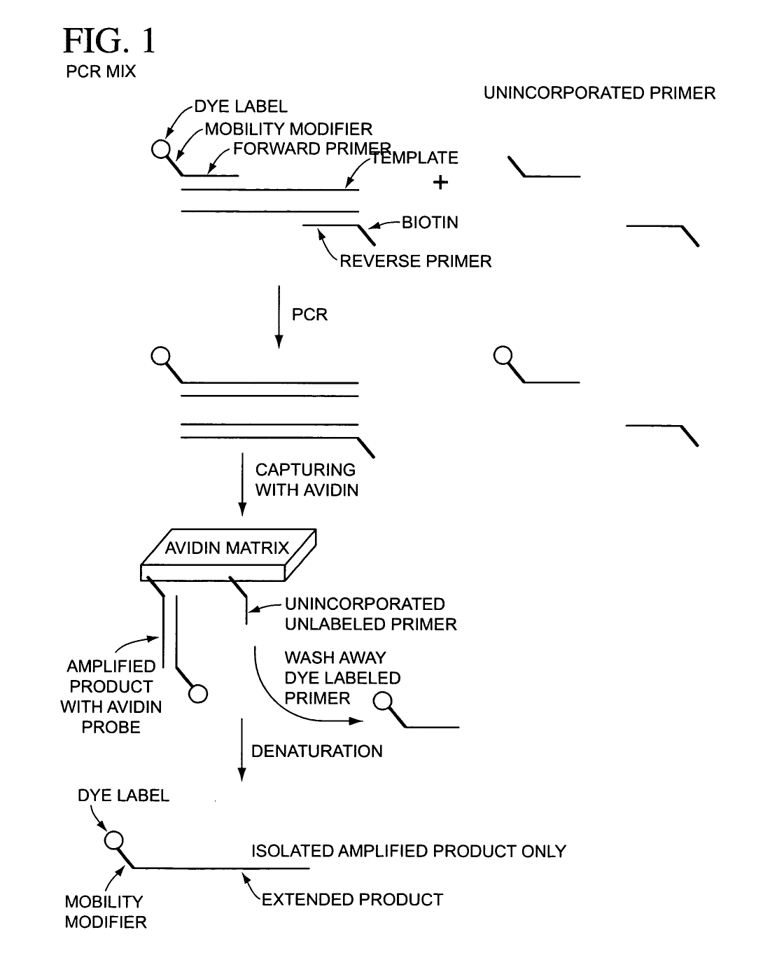

FIG. 2 shows a representative electropherogram in accordance with some embodiments of the present teachings. The traces represent results for experiments performed on a 3100 capillary electrophoresis platform (Applied Biosystems). The top trace is a PCR product mixture. The unincorporated primer peaks are at the 0 to 50 base pair region. The amplified product is at the 140-160 base pair region. The middle trace shows peaks representing unincorporated primers. The bottom trace shows uncontaminated peaks representing the isolated target strand of the PCR product.

FIG. 3 shows a schematic of a separation procedure in accordance with some embodiments of the present teachings.

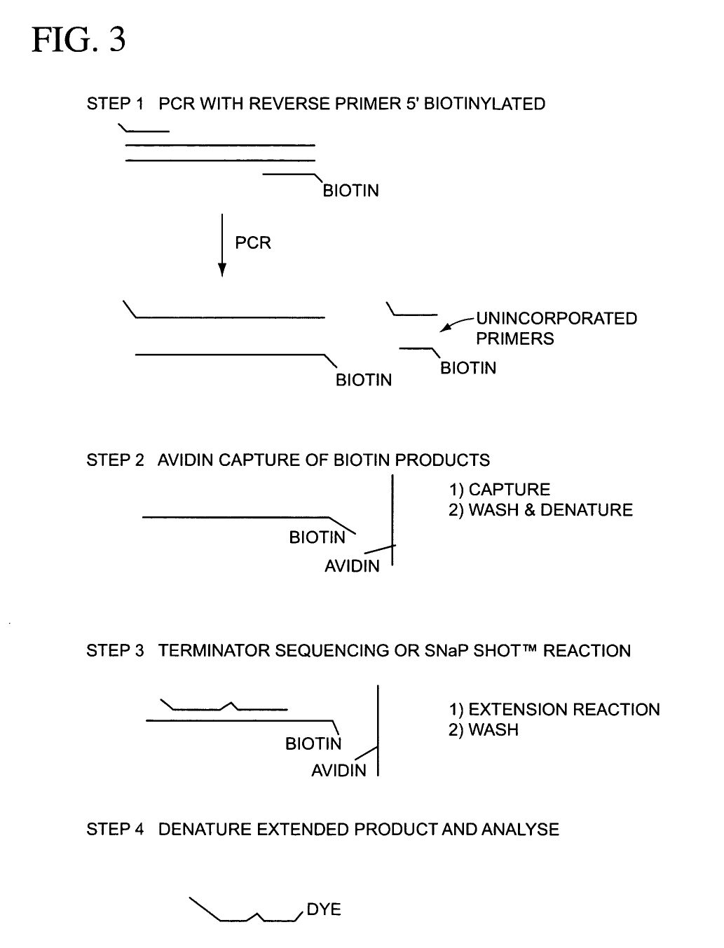

### DETAILED DESCRIPTION OF VARIOUS EMBODIMENTS

Numerous nucleic acid assays in the field of molecular biology involve complex reaction mixtures wherein the analysis of multiple genetic loci is to be performed. The present teachings involve the incorporation of at least one affinity moiety into at least one primer in a nucleic acid primer extension reaction, thereby facilitating separation, purification, identification, and analysis of complex mixtures of nucleic acids.

In some embodiments of the present teachings, primers in an amplification reaction can comprise affinity moieties, labels, and mobility modifiers that can facilitate analysis of resulting amplification products. For example, one primer of a primer pair flanking a polynucleotide region of interest can comprise an affinity moiety, and the other primer can comprise a label and a mobility modifier. The double stranded amplification product can be bound to an affinity-binding moiety, the unbound unincorporated reaction components removed, and the amplified target strand bearing the label and mobility modifier released and analyzed.

Some embodiments of the present teachings can be applied to the multiplexed analysis of degraded and/or non-degraded DNA in human forensics, wherein the target nucleic acids further comprise different polymorphic microsatellites that can be used to determine human identity. Some embodiments of the present teachings can be applied to the generation of molecular standards of specified sizes in a manufacturing setting. Some embodiments of the present teachings can be used in single nucleotide extension reactions in the context of identifying nucleic acid polymorphisms. Some embodiments of the present teachings can be used to improve the efficiency of electrokinetic injection.

The methods of the present teachings are also useful in such application as animal breeding, pedigree analysis, and livestock tracking generally. The methods of the present teachings can also be applied in an agricultural setting for plant identification and lineage analysis, as well as for the determination of genetic modification (ie status as genetically modified organism (GMO)). The methods of the present teachings are also useful in such application as genetic mapping (linkage analysis), paternity testing, and species identification (see for example U.S. Pat. No. 5,874,217), individual identification (see for example Tully et al., 1999, Tully et al., 1996, Ross et al., 1997) and extinction monitoring.

Primers in the extension reaction can be positioned to be complementary to and flank at least one polynucleotide region of interest present in at least one genome. For example, in the analysis of microsatellites, including those used in a human identification forensics context, primers can flank polynucleotide regions of interest that comprise one or more short tandem repeat regions. Analysis of the resulting amplicons can allow for the multiplexed detection of fragments that can be used to determine human identity. In some embodiments of the present teachings in a human identification forensics context, the primers can directly abut known polymorphic regions of the genome, thereby allowing for multiplexed extension reactions. In some embodiments of the present teachings in a human identification forensics context, the primers can nearly abut known polymorphic regions of the genome, thereby allowing for multiplexed extension reactions. In some embodiments nearly abutting primers can be as few as one nucleotide away from the start of a microsatellite region. In some embodiments nearly abutting primers can be more than one nucleotide away from the start of a microsatellite region. In some embodiments, primers can be placed several nucleotides away from the start of a microsatellite region. Primer selection can be optimized to provide for short fragments that are easy to amplify and/or provide for improved discriminatory capacity of the at least one polynucleotide region of interest.

A characteristic of multiplexed amplification reactions of polymorphic microsatelites is that amplicon size for a given polynucleotide region of interest can vary according to the individual organism from which a nucleic acid sample is collected. For example inter-individual variation in the unit repeat number for a polymorphic microsatellite comprising a polynucleotide region of interest can produce a plurality of different possible amplicon product lengths. As a result of this polymorphic variation, it can be difficult to know a priori the amplicon size resulting from a given target polynucleotide region of interest. Primer selection can be optimized to facilitate fragment identification in a multiplexed reaction, thereby increasing the number of identifiable fragments resulting from a single reaction and minimizing fragment overlap on a mobility dependent analysis technique.

There are a number of ways of manipulating primer design in order to facilitate identification of a plurality of amplicons in a multiplexed reaction. For example, in a multiplexed reaction involving the amplification of a plurality of polymorphic microsatellites, primers can be positioned so as to minimize size overlap of the resulting amplicons when analyzed on a mobility dependent analysis technique, for example capillary electrophoresis. Also, the primers can be chosen to amplify the plurality of polymorphic microsatellites in such fashion as to ensure non-lapping peaks on an electropherrogram-based readout. It will be appreciated that such optimizations can take into account the diversity of amplicon lengths for a given polynucleotide region of interest (conferred for example by variation in the number of repeat units) that could exist across the population of a given species. In a reaction context in which more than one polynucleotide region of interest occurs with potentially similar and overlapping amplicon sizes, criteria for selection of primers can include, for example, primers immediately abutting a polymorphic microsatellite at one locus, whereas primers for another locus of similar sequence length can be further away from the polymorphic microsatellite.

Another way of manipulating primer design in order to facilitate identification of a plurality of amplicons in a multiplexed reaction includes amplification of a plurality of polymorphic microsatellites using primers comprising mobility modifiers. Such mobility modifiers can be chosen and paired with primers in such fashion so as to minimize size overlap of similarly sized polymorphic microsatellites. Some embodiments of the present teachings comprise multiplexed reactions in which certain of the primer pairs comprise a mobility modifier, such that the migration rate in a mobility dependent analysis technique is conferred in part by the mobility modifier. Some embodiments of the present teachings involve multiplexed reactions in which certain of the primer pairs lack a mobility modifier, such that the migration rate in a mobility dependent analysis technique is largely imparted by the length of the amplified sequence. Some embodiments of the present teachings involve multiplexed reactions in which certain of the primer pairs lack a mobility modifier certain polynucleotide regions of interests, and certain of the primer pairs comprise a mobility modifer for other polynucleotide regions of interest, such that the migration rate in a mobility dependent analysis technique is imparted predominantly by the length of the amplified sequence for some amplicons, and imparted predominantly by the mobility modifier in some amplicons, and imparted by both the length of the amplified sequence and the mobility modifier in some amplicons.

The mobility modifier may be any entity capable of effecting a particular mobility of a single stranded target polynucleotide in a mobility-dependent analysis technique. In some embodiments, the mobility modifier can (1) have a low polydispersity in order to effect a well-defined and easily resolved mobility, e.g., Mw/Mn less than 1.05; (2) be soluble in an aqueous medium; (3) not adversely affect primer binding to the polynucleotide region of interest; and (4) be available in sets such that members of different sets impart distinguishable mobilities to the one or more single stranded target polynucleotides.

In one embodiment of the present teachings, the mobility modifier comprises a polymer. Specifically, the polymer may be homopolymer, random copolymer, or block copolymer. Furthermore, the polymer may have a linear, comb, branched, or dendritic architecture. In addition, although the present teachings are described herein with respect to a single polymer chain attached to an associated mobility modifier at a single point, the present teachings also contemplate mobility modifiers comprising more than one polymer chain element, where the elements collectively form a mobility modifier.

In some embodiments, polymers for use in the present teachings are hydrophilic, or at least sufficiently hydrophilic when bound to a primer to ensure it is readily soluble in aqueous medium. The polymer should also not affect the hybridization between a primer and a polynucleotide region of interest. Where the primer is charged and the mobility-dependent analysis technique is electrophoresis, the polymers can be uncharged or have a charge/subunit density that is substantially less than that of the primer.

In one embodiment, the polymer is polyethylene oxide (PEO), e.g., formed from one or more hexaethylene oxide (HEO) units, where the HEO units are joined end-to-end to form an unbroken chain of ethylene oxide subunits. Other exemplary embodiments include a chain composed of N 12mer PEO units, and a chain composed of N tetrapeptide units, where N is an adjustable integer (e.g., Grossman et al., U.S. Pat. No. 5,777,096).

Clearly, the synthesis of polymers useful as a mobility modifier of the present teachings will depend on the nature of the polymer. Methods for preparing suitable polymers generally follow well-known polymer subunit synthesis methods. Methods of forming selected-length PEO chains are well-known, and involve coupling of defined-size, multi-subunit polymer units to one another, either directly or through charged or uncharged linking groups, are generally applicable to a wide variety of polymers, such as polyethylene oxide, polyglycolic acid, polylactic acid, polyurethane polymers, polypeptides, and oligosaccharides. Such methods of polymer unit coupling are also suitable for synthesizing selected-length copolymers, e.g., copolymers of polyethylene oxide units alternating with polypropylene units. Polypeptides of selected lengths and amino acid composition, either homopolymer or mixed polymer, can be synthesized by standard solid-phase methods (e.g., Fields and Noble, Int. J Peptide Protein Res., 35: 161-214 (1990)).

In one method for preparing PEO polymer chains having a selected number of HEO units, an HEO unit is protected at one end with dimethoxytrityl (DMT), and activated at its other end with methane sulfonate. The activated HEO is then reacted with a second DMT-protected HEO group to form a DMT-protected HEO dimer. This unit-addition is then carried out successively until a desired PEO chain length is achieved (e.g., Levenson et al., U.S. Pat. No. 4,914,210).

Another polymer for use as a mobility modifier in the present teachings is PNA (peptide nucleic acid). In particular, when used in the context of a mobility-dependent analysis technique comprising an electrophoretic separation in free solution, PNA has the advantageous property of being essentially uncharged.

Coupling of the polymer to a primer can be carried out by an extension of conventional phosphoramidite polynucleotide synthesis methods, or by other standard coupling methods, e.g., a bis-urethane tolyl-linked polymer chain may be linked to an polynucleotide on a solid support via a phosphoramidite coupling. Alternatively, the polymer chain can be built up on a polynucleotide (or other tag portion) by stepwise addition of polymer-chain units to the polynucleotide, e.g., using standard solid-phase polymer synthesis methods. As noted above, the mobility modifier imparts a mobility to a primer that can be distinctive for a polynucleotide region of interest. The contribution of the mobility modifier to the mobility of the single stranded target polynucleotide will in general depend on the size of the mobility modifier. However, addition of charged groups to the tail, e.g., charged linking groups in the PEO chain, or charged amino acids in a polypeptide chain, can also be used to achieve selected mobility characteristics in the single stranded target polynucleotide. It will also be appreciated that the mobility of a single stranded target polynucleotide can be influenced by the properties of the primer itself, e.g., in electrophoresis in a sieving medium, a larger primer sequence will reduce the electrophoretic mobility of a given single stranded target polynucleotide as compared to a shorter primer.

For illustrative mobility modifiers, and methods of synthesis, see U.S. Pat. No. 5,514,543, U.S. Pat. No. 5,470,705, U.S. Pat. No. 5,580,732, U.S. Pat. No. 5,624,800, U.S. Pat. No. 5,807,682, PCT Publication No. WO 01/92579, and U.S. application Ser. No. 09/836,704, which are hereby expressly incorporated by reference in their entirety.

Another way of manipulating primer design in order to facilitate identification of a plurality of amplicons involves the use of labels with primers to provide additional amplicon identification and determination. For example, when amplicons resulting from two different polynucleotide regions of interest can possibly comprise overlapping sizes, the primers amplifying the different polynucleotide regions of interest can comprise distinct labels, thereby allowing for their separate identification.

It will be appreciated that the present teachings contemplate using the collection of these parameters (primer placement, presence or absence of a mobility modifier on a primer, type and composition of mobility modifier, length of amplicon sequence, label, and the like) in such fashion as to optimize a multiplexed reaction in order to facilitate the identification of plurality of reaction products. It will be appreciated that many of these parameters manipulated for primer design can be employed in multiplexed reactions involving polymorphic microsatellites, for example in the field of forensics and human identification. It will further be appreciated that many of these parameters manipulated for primer design can also be employed in multiplexed reactions not involving polymorphic microsatellites, for example single nucleotide extension reactions, etc. It will be appreciated that some embodiments of the present teachings can be applied in the area of forensic science wherein samples can be degraded, such that removal of unincorporated reaction components, as well as primer compositions of the present teachings, can provide for increased numbers of identifiable amplicons as assessed by a mobility dependent analysis technique. In some embodiments, it will be appreciated that the increased numbers of identifiable amplicons assessed by a mobility dependent analysis technique reside in those regions of analytic space in an electropherrogram that might otherwise be occupied by unincorporated primers and other unincorporated reaction components.

Primers can comprise an affinity moiety, thereby allowing for the binding of reaction products to affinity-binding moieties. For example, a specific binding pair comprising biotin and streptavidin can be employed. A biotin affinity moiety can be incorporated into a primer, and a streptavidin binding moiety used to bind, or bind and immobilize, the resulting reaction product. Unbound unincorporated reaction components can be removed, and the strand complementary to the biotin-bearing strand isolated and analyzed. As used herein, such strands will be referred to as “affinity moiety strand” and “labeled single stranded target polynucleotide.” It will be appreciated that the members of a specific binding pair can be switched without straying from the scope of the present teachings, wherein for example the streptavidin is attached to the primer and acts as an affinity moiety, and the biotin is attached to a solid support and acts as a binding moiety. Further, the procedures used for binding, and/or binding and immobilization, of the affinity strand are numerous to one of skill in the art. For illustrative examples, see inter alia Hermanson, Bioconjugate Techniques, 1996).

It will be appreciated that the present teachings include primer modifications known in the art to optimize reaction parameters, such as melting temperature in order to manipulate stringency. For example, the primers can comprise nucleotide analogs such as LNA, PNA, and/or INA. It will also be appreciated that the present teachings consider multiplexed reactions in which certain of the primer pairs further comprise a mobility modifier, while other primer pairs do not comprise a mobility modifier. Furthermore, in some embodiments, primers can comprise regions of non-complementarity with the target nucleic acids, which in some embodiments can impart mobility information to the resulting reaction product.

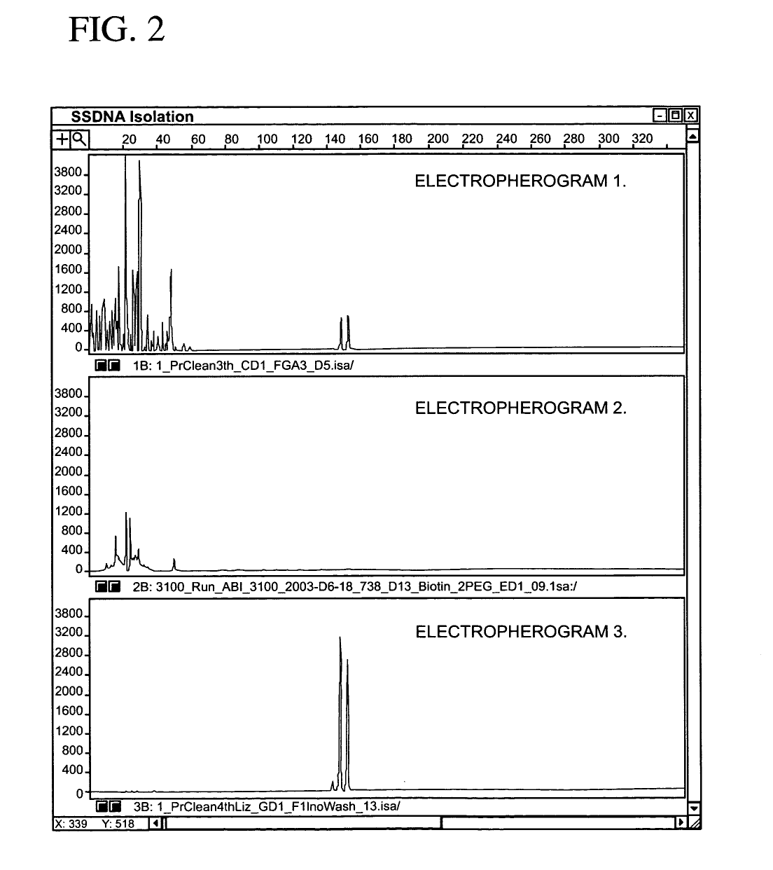

In some embodiments, the present teachings relate to multiplexed reactions, wherein multiple polynucleotide regions of interest are analyzed. In multiplexed amplification reactions, numerous primer pairs that flank different polynucleotide regions of interest can be employed. Unincorporated reaction components in a multiplexed reaction can unnecessarily complicate analysis of reaction products. For example, in the context of capillary electrophoresis, a multiplexed amplification reaction can produce a plurality of peaks, the identification of which requires a certain analytic range on an electrophorrogram. Unincorporated reaction components can unnecessarily occupy and interfere with a portion of this analytical range, rendering it unable or difficult to provide information regarding target identity (see FIG. 2). Removal of unincorporated reaction components from the reaction mixture allows for smaller reaction products to be analyzed in this portion of the electrophoretic analytic range. In some embodiments, removal of unincorporated reaction components can eliminate or reduce the amount of unincorporated reaction components that co-migrate near the amplicons, thereby facilitating the ability to distinguish signal peaks resulting from desired amplicons versus background peaks resulting from unincorporated reaction components. In some embodiments, the present teachings provide a greater degree of assay design flexibility in a multiplexed setting, whereby primer pairs flanking target nucleic acids can be chosen with the flexibility to position the primers to produce products of size convenient to maximize information extraction. In some embodiments of the present teachings, PCR reactions as described herein are employed to amplify fragments from at least one microsatellite region. In some embodiments of the present teachings, the fragments are amplified with a primer comprising a fluorophore and a mobility modifier, and/or with a hybridization enhancer (e.g., a minor groove binder). Where more than one microsatellite region is to be amplified, detectable fluorophore and mobility modifiers are selected such that different amplicons are readily distinguished. As an example, different colored fluorophores can be used to analytically distinguish different microsatellites, wherein amplicon lengths overlap between the two polynucleotide regions of interest. Furthermore, the same color fluorophore can be used to amplify fragments containing microsatellites that generate fragments of different sizes that are thereby readily discernable, for example by electrophoretic separation.

The present teachings provide amplification of target nucleic acids, with detection resulting from the increased amount of target relative to the copy number present in the starting material. Suitable amplification procedures include the polymerase chain reaction, although it will be appreciated that other amplification strategies might be employed in order to generate enough product for detection. In some embodiments, the present teachings contemplate labels of sufficient intensity so as to obviate an amplification step, for example the incorporation of various Quantum Dots into the nucleotides of a multiplexed single nucleotide primer extension reaction (see Xu et al., Nucleic Acids Res. 2003 Apr 15;31(8):e43.).

The enzyme that polymerizes the nucleotide triphosphates into the amplified fragments of the PCR may be any DNA polymerase, including heat-resistant polymerases known in the art. Polymerases that may be used in the invention include, but are not limited to DNA polymerases from such organisms as Thermus aquaticus, Thermus thermophilus, Thermococcus litoralis, Bacillus stearothermophilus, Thermotoga maritima and Pyrococcus ssp. The enzyme may be isolated from the source bacteria, produced by recombinant DNA technology or purchased from commercial sources. For example, DNA polymerases are available from Applied Biosystems and include AmpliTaq Gold® DNA polymerase; AmpliTaq® DNA Polymerase; Stoffel fragment; rTth DNA Polymerase; and rTth DNA Polymerase XL. Other suitable polymerases include, but are not limited to Tne, Bst DNA polymerase large fragment from Bacillus stearothermophilus, Vent and Vent Exo- from Thermococcus litoralis, Tma from Thermotoga maritima, Deep Vent and Deep Vent Exo- and Pfu from Pyrococcus, and mutants, variants and derivatives of the foregoing. For further discussion of polymerases, and applicable molecular biology procedures generally see, Ausubel et al., CURRENT PROTOCOLS IN MOLECULAR BIOLOGY, John Wiley & Sons, New York, 2001, THE POLYMERASE CHAIN REACTION, Mullis, K. B., F. Ferre, and R. A. Gibbs, Eds., MOLECULAR CLONING: A LABORATORY MANUAL (3rd ed.) Sambrook, J. & D. Russell, =Eds. Cold Spring Harbor Laboratory Press, Cold Spring Harbor, N.Y. (2001), and Wojciechowski et al., 1999.

Amplification reaction times, temperatures and cycle numbers may be varied to optimize a particular reaction as a matter of routine experimentation. Further, the addition of additives to reduce stutter and reduce non-specific amplification are further contemplated, as discussed in U.S. application Ser. No. 09/850,514 and U.S. application Ser. No. 09/850,590, and U.S. application Ser. No. 09/998887. In some embodiments of the present teachings, it is advantageous to incubate the reactions at a certain temperature following the last phase of the last cycle of PCR. In some embodiments, a prolonged extension phase is selected. In other embodiments, an incubation at a low temperature (e.g., 4° C.) is selected.

Following amplification and/or labeling of the target nucleic acids, the affinity moiety can be bound to the binding moiety. Numerous affinity-moiety binding interaction procedures are known in the art, see for example commercial products from Pierce, Millipore, Roche, Magnetic Solutions, Hydros Inc., and Beckman. For example, streptavidin coated magnetic beads can be used to immobilize biotin labeled amplification products. In another example, streptravidin plates can be used to immobilize biotin labeled amplification products. Following binding to the binding moiety, unbound unincorporated reaction components can be removed by washing. The labeled single stranded target polynucleotide can then be removed, and analyzed. In some embodiments, the labeled single stranded target polynucleotide is removed by denaturation, such denaturation procedures including heat, alkali, decreasing salt concentration, varying voltage, (see for example U.S. Pat. No. 6,124,092, U.S. Pat. No. 6,207,818, and Published U.S. application Ser. No. 09/908,994) and other methods well known in the art.

In some embodiments of the present teachings, primers in an amplification reaction can further comprise restriction enzyme recognition sequences that can facilitate analysis of resulting amplification products. For example, one primer of a primer pair flanking a target nucleic acid can comprise an affinity moiety, and the other primer can comprise a label, a mobility modifier, and a restriction enzyme recognition sequence. The double stranded amplification product can be bound to an affinity-binding moiety, the unbound unincorporated reaction components removed, and a portion of the amplified strand comprising the label and mobility modifier released by restriction endonuclease digestion, wherein the restriction enzyme recognizes restriction enzyme sites incorporated into the primer comprising the label and the mobility modifier. The products resulting from such restriction nuclease treatment can then undergo a mobility dependent analysis technique, and the identity of the polynucleotide region of interest determined therefrom. In some embodiments, the primer that comprises the restriction enzyme recognition sequence can comprise sequence that can or cannot hybridize to a region flanking a target polynucleotide region of interest and provide for its amplification. In such fashion, the primer can allow the eventual presence of the cleaved product to indicate presence of the target polynuceotide region of interest in the sample.

In some embodiments, a primer directly abutting a polymorphic site can be used in a single nucleotide primer extension reaction. A single nucleotide extension reaction can comprise treating a sample containing the target sequence of interest in single stranded form with a complementary primer under hybridization conditions such as to form a duplex, contacting the duplex with at least two labeled nucleotide terminators, extending the primer wherein one of the terminators is complementary to a nucleotide base to be identified, and determining the presence and identity of the nucleotide base at the specific position in the nucleic acid of interest by detecting the label (for example see U.S. Pat. No. 5,888,819, and Orchid GeneScreen). In some embodiments of the present teachings, a primer in a single nucleotide reaction can further comprise a mobility modifier, and the extended nucleotide can further comprise a label. In some embodiments, affinity moieties can be incorporated into the target nucleic acids, thereby allowing interaction with binding moieties. Primers can be hybridized to immobilized target nucleic acids, single nucleotide extension performed, and isolated extension products can be analyzed and the identity of the labeled single stranded target polynucleotide determined based on the information conveyed by the primer, mobility modifier, and/or the label. For exemplary mobility modifiers and labels, see infra. In some embodiments of the present teachings, a whole genome amplification is performed, and this amplified whole genome can serve as the substrate for a single nucleotide extension reaction. The products of the single nucleotide extension reaction can then be analyzed. In some embodiments, the whole genome amplification can further comprise the introduction of biotin, or other affinity moieties. In some embodiments, the single nucleotide extension reaction can be performed subsequent to an amplification reaction in which at least one polynucleotide region of interest is amplified, wherein the primers hybridize and amplify the region regardless of the polymorphic nucleotide contained therein, and the eventual single nucleotide extension reaction allows for the eventual elucidation of the polymorphic nucleotide. For methods for amplifying a plurality of polynucleotide regions of interest see for example U.S. Pat. No. 6,605,451.

Some embodiments of the present teachings comprise a single base extension reaction for single nucleotide polymorphism detection, wherein the primer can bear a mobility modifier, the nucleotides can bear a label, and the target nucleic acids can bear an affinity moiety. In some embodiments, the sample and the polynucleotide regions of interest can be biotinylated using photo-biotin and immobilized (see for example, Hermanson, 1996). Hybridization of a primer comprising a mobility modifier, and performing a single nucleotide extension reaction with a labeled nucleotide and a polymerase, can result in a labeled single stranded target polynucleotide. Release and analysis of the labeled single stranded target polynucleotide can result in determination of the polynucleotide region of interest.

In some embodiments comprising single nucleotide extension reactions, a primer bears an affinity moiety, thereby allowing an affinity binding moiety to bind the amplified nucleic acids on the sample, and the unhybridized, non-extended, unincorporated reaction components can be removed. Hybridization of a primer bearing a mobility modifier, and extension of a labeled nucleotide with a polymerase, results in a reaction product strand bearing the label and mobility modifier. Release of the labeled single stranded target polynucleotide and subsequent analysis can result in the determination of the polynucleotide region of interest.

In some embodiments, the labeled single stranded target polynucleotides undergo electrokinetic injection in the process of capillary electrophoresis analysis. Such injections are influenced by the levels of salt in the samples, wherein the amount of DNA injected is inversely proportional to the ionic strength of the sample (see Belgrader et al., 1996, Ruiz-Martinez et al., 1998, Salas-Solano et al., 1998). In some embodiments of the present teachings, removal of unincorporated reaction components also results in the removal of salt from the reaction mixture, thereby resulting in target nucleic acids with reduced salt levels used in the electrokinetic injection and allowing more target nucleic acid strand to be loaded per unit volume as a result. Further, by reducing the amount of DNA sample needed for each capillary, less amplified target nucleic acids, and/or less original starting material can potentially be used.

In some embodiments, a mobility-dependent analytical technique (MDAT) is used to analyze the labeled single stranded target polynucleotidides. Exemplary mobility-dependent analysis techniques include electrophoresis, chromatography, mass spectroscopy, sedimentation, e.g., gradient centrifugation, field-flow fractionation, multi-stage extraction techniques and the like. Descriptions of mobility-dependent analytical techniques can be found in, among other places, U.S. Pat. Nos. 5,470,705, 5,514,543, 5,580,732, 5,624,800, and 5,807,682 and PCT Publication No. WO 01/92579.

The amplification products can be analyzed in on a sieving or non-sieving medium. Amplification reactions can also be analyzed by denaturing samples and separating using a capillary electrophoresis protocol in an ABI PRISMS® 310 genetic analyzer, or by separating on a 4.5%, 29:1 acrylamide:bis acrylamide, 8 M urea gel prepared for an ABI 377 Automated Fluorescence DNA Sequencer, or by higher throughput florescence-based automated capillary electrophoresis instruments such as the ABI 3100, ABI 3700, and ABI 3730×1. Sequence data may be analyzed with GeneScan Software from Applied Biosystems. In some embodiments of the present teachings, for example, the PCR products are analyzed by capillary electrophoresis as described in Wenz, H. et al. (1998) Genome Res. 8:69-80. In some embodiments of the present teachings, for example, the PCR products are analyzed by slab gel electrophoresis as described in Christensen, M. et al. (1999) Scand. J Clin. Lab. Invest. 59(3):167-177. Fragments may be analyzed by chromatography (e.g., size exclusion chromatography (SEC)).

In the area of forensic science, identification of human remains can be hindered by degraded DNA samples (Butler et al., 2003, Grubwieser et al., 2003, Wiegand et al., 2001, Tsukada et al., 2002, Hellmann et al., 2001). It has been shown that despite extensive degradation, nucleic acids comprising a few hundred nucleotides can nonetheless routinely be amplified from extensively degraded source material. As a result, forensic identification can be better achieved in degraded source material by targeting microsatellites variants of smaller unit length, those smaller unit lengths that have fewer repeat units in all known allelic variants, and/or by amplifying such regions with immediately flanking primers. These approaches amplify small fragments, thereby increasing the likelihood that the small polynucleotide region of interest will remain intact in the degraded sample. However, such an approach is difficult to multiplex with different loci of interest since all loci will have similar electorophoretic migration profiles, and hence interpretation of the resulting data and peak identification problematic. The present teachings help address this issue by both providing for the analysis of actual desired amplification products bearing mobility modifiers, and analyzing products in that portion of the electrophoretic read-out space that would otherwise potentially be occupied by unincorporated reaction components, for example labeled unincorporated primers. In some embodiments of the present teachings, degraded DNA fragments are in the range of 60-240 nucleotides. In some embodiments of the present teachings, degraded DNA fragments are in the range of 20-60 nucleotides. In some embodiments of the present teachings, degraded DNA fragments are in the range of 60-100 nucleotides. In some embodiments of the present teachings, degraded DNA fragments are in the range of 100-140 nucleotides. In some embodiments of the present teachings, degraded DNA fragments are in the range of 140-180 nucleotides. In some embodiments of the present teachings, degraded DNA fragments are in the range of 180-220 nucleotides. In some embodiments of the present teachings, degraded DNA fragments are in the range of 220-240 nucleotides.

In some embodiments, as well as in instances of severe sample degradation, it can be desirable to amplify and detect single nucleotide polymorphisms rather than microsatellites. For example, single nucleotide primer extension reactions require less intact DNA sequence and can also be multiplexed. Interpretation of data resulting from single nucleotide primer extension reactions can be complicated by unincorporated reaction components. Some embodiments of the present teachings help address this issue by providing removal of unincorporated reaction components. The present teachings help address this issue by both providing for the analysis of actual desired amplification products bearing mobility modifiers, and analyzing products in that portion of the electrophoretic read-out space that would otherwise potentially be occupied by unincorporated reaction components, for example labeled unincorporated primers

In some embodiments of the present teachings in a human forensics application, amplification of the following marker loci is performed: THO1, AMG, D8, FGA, D3, D16, D18, TPOX, CSF, D19, D21, D7, D5, D13, D2, vWA. Also see http:/Hwww.csti.nist.gov/biotech/strbase/ for other relevant loci included in some embodiments of the present teachings.

In the field of human identity, tetranucleotide microsatellites can be used in forensic casework, establishment of convicted felon databases, disaster and military victim identification (Fre'geau et al. (1993) Biotechniques 15:100-119). Furthermore, they have proved useful in forensics to identify human remains. In the analysis of museum specimens and in parentage testing. Tetranucleotide microsatellites are specifically powerful in these applications, since multiple microsatellite tests that have matching probabilities of one in several billion individuals are now available. Examples of microsatellite containing alleles which can be used for paternity, forensic and other personal identification include but are not limited to D3S1358; VWA; D16S539; D8S1179; D21S11; D18S51; D19S433; THOI; FGA; D7S820; D13S317; D5CSFIPO; TPOX. Genotyping methods used for human identification may also be applied to plant and animal breeding, using appropriate genetic loci.

Personal identification tests can be performed on any specimen that contains nucleic acid such as bone, hair, blood, tissue and the like. DNA may be extracted from the specimen and a panel of primers to amplify a set of microsatellites used to amplify DNA to generate a set of amplified fragments. In forensic testing, the specimen's microsatellite amplification pattern is compared with a known sample from the presumptive specimen or is compared to the pattern of amplified microsatellites derived from the presumptive specimen's family members (e.g., the mother and father) wherein the same set of microsatellites are amplified and the resulting target nucleic acid strand isolated. The pattern of microsatellite amplification may be used to confirm or rule out the identity of the specimen. In paternity testing, the specimen is generally from the child and the comparison is made to the microsatellite pattern from the presumptive father, and may include matching with the microsatellite pattern from the child's mother. The pattern of microsatellite amplification may be used to confirm or rule out the identity of the father. The panel can include microsatellites with a G+C content of 50% or less such as, for example, D3S1358; vWA; D16S539; D8S1179; D21S11; D18S51; D19S433; TH01; FGA; D7S820; D13S317; D5S818; CSF1PO; TPOX; hypozanthine phosphoribosyltransferase; intestinal fatty acid-binding protein; recognition/surface antigen; c-fms proto-oncogene for CFS-1 receptor; tyrosine hydroxylase; pancreatic phospholipase A-2; coagulation factor XIII; aromatase cytochrome P-450; lipoprotein lipase; c-fes/fps proto-oncogene; and unknown fragment. Isolation of target nucleic strands in accordance with the present teachings is useful the above applications, which are illustrative and not limiting. The present teachings further contemplate the detection of chimerism using the methods and compositions and kits described herein.

The interpretation of data provided by the methods of the present teachings and can be applied to a variety of contexts. The methods of the present teachings may be used in conjunction with the methods described in the references cited herein, the disclosure of each of which is incorporated herein by reference in its entirety. In some embodiments of the present teachings, the methods will simplify analyses of forensic samples, and therefore can find particular utility in the field of forensics.

Definitions

As used herein, the term label refers to any moiety that, when attached to a nucleotide or polynucleotide, renders such nucleotide or polynucleotide detectable using known detection methods. Labels may be direct labels which themselves are detectable or indirect labels which are detectable in combination with other agents. Exemplary direct labels include but are not limited to fluorophores, chromophores, radioisotopes (e.g., ³²P,³⁵S,³H), spin-labels, Quantum Dots, chemiluminescent labels, and the like. Exemplary indirect labels include enzymes which catalyze a signal-producing event, and ligands such as an antigen or biotin which can bind specifically with high affinity to a detectable anti-ligand, such as a labeled antibody or avidin. Many comprehensive reviews of methodologies for labeling DNA provide guidance applicable to the present invention. Such reviews include Matthews et al. (1988); Haugland (1992), Keller and Manak (1993); Eckstein (1991); Kricka (1992), and the like.

As used herein, the term “affinity moiety” refers to a molecular composition capable of selective interaction with a cognate binding moiety, such as for example biotin/avidin, ligand/receptor, and the like. Detailed protocols for methods of attaching binding moieties to oligonucleotides can be found in, among other places, G. T. Hermanson, Bioconjugate Techniques, Academic Press, San Diego, Calif. (1996) and S. L. Beaucage et al., Current Protocols in Nucleic Acid Chemistry, John Wiley & Sons, New York, N.Y. (2000).

As used herein, the term “affinity moiety strand” refers to a strand resulting from the PCR in which the affinity moiety is incorporated by the presence of the affinity moiety in the primer.

As used herein, the term “binding moiety” refers to a molecular composition capable of selective interaction with a cognate affinity moiety, such as for example biotin/avidin, ligand/receptor, and the like.

As used herein, the term “unbound unincorporated reaction components” refers to those components of the PCR that are not incorporated into the double stranded polynucleotide amplification product, and that are not bound to the binding moiety, such components including unincorporated primers lacking the affinity moiety, nucleotides, enzyme, and buffer components.

As used herein, the term “denaturation” refers to separation of complementary strands of DNA, which can be achieved through a number of methods such as heat, alkali, voltage, and other procedures known in the art to disrupt Watson-Crick hydrogen bonding between complementary DNA strands.

As used herein, the term “degraded DNA” refers to DNA that has undergone deterioration as a result of time, temperature, environmental conditions, and the like, resulting in a reduction of fragment size. It will be appreciated that DNA can be both damaged and degraded, and that use of the term degraded DNA is not exclusive of damaged DNA.

As used herein, the term “damaged DNA” refers to DNA that has undergone deterioration as a result of time, temperature, environmental conditions, and the like, resulting in a loss of base information. It will be appreciated that DNA can be both damaged and degraded, and that use of the term damaged DNA is not exclusive of degraded DNA.

As used herein, term “sample” refers to the source material that comprises the polynucleotide regions of interest, and from which the labeled target single stranded polynucleotide is eventually amplified.

As used herein, the term “molecular standard” refers to fragments of DNA of known length.

As used herein, the term “polymorphic microsatellite” refers to a genetic locus comprising a short (e.g., 1-6 or more nucleotide), tandemly repeated sequence motif. As used herein the term microsatellite is synonymous with short tandem repeat (STR). As used herein “mononucleotide microsatellite” refers to a genetic locus comprising a repeated nucleotide (e.g., A/T). “Dinucleotide microsatellite” refers to a genetic locus comprising a motif of two nucleotides that is tandemly repeated (e.g., CA/TG, CT/GA). “Trinucleotide microsatellite” refers to a genetic locus comprising motif of three nucleotides that is tandemly repeated (e.g., GAA/TTC). “Tetranucleotide microsatellite” refers to a genetic locus comprising a motif of four nucleotides that is tandemly repeated (e.g., TCTA/TAGA, AGAT/ATCT, AGAA/TTCT, AAAG/CTTT, AATG/CATT, TTTC/GAAA, CTTT/AAAG and GATA/TATC). “Pentanucleotide microsatellite” refers to a genetic locus comprising a motif of five nucleotides that is tandemly repeated (e.g., AAAGA/TCTTT). Microsatellites may contain repeat-motif interspersions, or “cryptically simple sequence” (Tautz, D. et al. (1986) Nature 322(6080):652-656). Such repeat-motif interspersions include simple repeat-motif interspersions wherein the microsatellite contains one or more interspersed repeats with the same length as the tandemly repeated sequence motif, but a different repeat sequence. For example, if the tandemly repeated sequence motif is TCTA, a simple repeat-motif interspersion may appear as follows: TCTA(TCTG)₂(TCTA)₃, wherein the interspersed repeat “TCTG” interrupts the repeat of the TCTA tandemly repeated sequence motif. Repeat-motif interspersions also include more complex repeat-motif interspersions wherein the repeat motif interspersion is not the same length as the tandemly repeated sequence motif. For example, if the tandemly repeated sequence motif is TCTA, the complex repeat-motif interspersion may appear as follows: (TCTA)₃TA(TCTA)₃TCA(TCTA)₂, wherein the tandemly repeated sequence motif is interrupted by TA and TCA. Other more complex repeat motif interspersions include the combination of the simple repeat-motif interspersion and the complex repeat-motif interspersion in the same microsatellite. For example, such a complex sequence repeat-motif interspersion may appear as follows: (TCTA)ₙ(TCTG)₀(TCTA)₃TA(TCTA)₃TCA(TCTA)₂TCCATA(TCTA)ₚ, wherein both forms of interspersed repeats interrupt the tandemly repeated sequence motif, TCTA. Microsatellites with and without interspersed repeats are encompassed by the term “microsatellites” as used herein.

The term “mobility modifier” as used herein refers to at least one polymer chain that when added to at least one reaction component that affects the mobility of the element to which it is bound in a mobility-dependent analytical technique. Typically, a mobility modifier changes the charge/translational frictional drag when to an element e.g. primer); or imparts a distinctive mobility, for example but not limited to, a distinctive elution characteristic in a chromatographic separation medium or a distinctive electrophoretic mobility in a sieving matrix or non-sieving matrix (see, supra, as well as e.g., U.S. Pat. Nos. 5,470,705 and 5,514,543, as well as U.S. application Ser. No. 09/836,704).

The term “mobility-dependent analytical technique” (MDAT) as used herein is a technique based on differential rates of migration between different species being separated. Exemplary mobility-dependent analysis techniques include electrophoresis, chromatography, mass spectroscopy, sedimentation, e.g., gradient centrifugation, field-flow fractionation, multi-stage extraction techniques and the like. Descriptions of mobility-dependent analytical techniques can be found in, among other places, U.S. Pat. Nos. 5,470,705, 5,514,543, 5,580,732, 5,624,800, and 5,807,682 and PCT Publication No. WO 01/92579.

The present teachings will be further described using the following example, which is merely illustrative of some embodiments of the present teachings. The examples should not be construed in any way to limit the scope of the invention, which is defined by the appended claims.

### EXAMPLE

A 25 ul PCR amplification comprises:
1 ng of template DNA
20 pmoles of each primer
9.55 ul of AmpLISTR® (PCR Reaction Mix (Applied Biosystems)
2.22 Units of AmpliTaq® Gold (Applied Biosystems).

PCR is performed with a PE Biosystems GeneAmp 9700 thermal cycler running in 9600 emulation mode under the following cycling conditions:
11 minutes at 95 C
28 cycles of 1 minute at 94, 1 min at 59 C, 1 min at 72 C;
60 minutes at 60 C.

The capturing and washing protocol comprises diluting 10 ul of the PCR product with 23 ul of 0.1×SSC Buffer. The product is added to avidin-coates (Strepta Well, Roche Diagnostics GmbH) and is rotated on a rotator for 40 minutes. The supernatant is then removed. The pellet is washed 4 times with 100 ul of 0.1×SSC, with centrifugation between the wash steps. 30 ul of 95 C HiDi Formamide is added to the washed pellet, and the target nucleic acid removed.

The target nucleic acid is then loaded onto a 3100 Genetic Analyzer (Applied Biosystems) with 1 ul of 500 Genescan Size Standard and run under the following conditions:
Filter Set: G5
Module: GeneScan36vb_POP4_G5module
Run Temperature: 60 C
Run Current: 100 uAmps
Injection Voltage: 3 kVolts
Injection Time: 10 seconds
Run Voltage: 15 kVolts
Run Time: 1500 seconds

1. A method for isolating a labeled single stranded target polynucleotide comprising, forming a polymerase chain reaction (PCR) mixture comprising,
a. a polynucleotide region of interest,
b. a first primer specific for the region of interest, wherein the primer has a label and a mobility modifier,
c. a second primer specific for the region of interest, wherein the second primer comprises an affinity moiety, thereby forming a reaction mixture,
amplifying the region of interest, thereby producing a double stranded polynucleotide amplification product comprising the labeled single stranded target polynucleotide comprising the label and the mobility modifier, and a complementary affinity moiety strand,
contacting the reaction mixture with a binding moiety specific for the affinity moiety,
binding the double stranded polynucleotide amplification product to the binding moiety,
removing the unbound unincorporated reaction components, and,
releasing the labeled single stranded target polynucleotide from the bound double
stranded polynucleotide amplification product by denaturation.

2. The method according to claim 1 wherein said mobility modifier is chosen from the group comprising nucleotides, polyethylene oxide, polyglycolic acid, polylactic acid, polypeptide, oligosaccharide, and polyurethane, polyamide, polysulfonamide, polysulfoxide, and block copolymers thereof, including polymers composed of units of multiple subunits linked by charged or uncharged linking groups, and combinations thereof.

3. The method according to claim 1 wherein the binding moiety is streptavidin.

4. The method according to claim 1 wherein the affinity moiety is biotin.

5. The method according to claim 1 wherein the PCR mixture further comprises a plurality of primer sets, each primer set comprising a first primer and a second primer flanking a region of interest, wherein the first primer further comprises the label and the mobility modifier, and wherein the second primer further comprises the affinity moiety

6. The method according to claim 5 wherein the polynucleotide region of interest is derived from a sample that further comprises degraded DNA.

7. The method according to claim 6 wherein said degraded DNA is between about 60 and 240 nucleotides in length.

8. The method according to claim 7 wherein the regions of interest further comprise polymorphic microsatellites.

9. The method according to claim 8 wherein the polymorphic microsatellites further comprise a dinucleotide repeat.

10. The method according to claim 8 wherein the polymorphic microsatellites further comprise a trinucleotide repeat.

11. The method according to claim 8 wherein the polymorphic microsatellites further comprise a tetranucleotide repeat.

12. The method according to claim 5 wherein at least one of the single stranded target polynucleotides results from amplification with a primer pair lacking a mobility modifier.

13. The method according to claim 1 wherein the PCR mixture further comprises sorbitol.

14. The method according to claim 1 wherein the PCR mixture further comprises betaine.

15. The method according to claim 1 wherein the PCR mixture further comprise sorbitol and betaine.

16. A method for manufacturing a labeled single stranded target polynucleotide molecular size standard comprising,
forming a PCR mixture comprising,
a. a polynucleotide region of interest,
b. a first primer specific for the region of interest, wherein the first primer comprises a label and a mobility modifier,
c. a second primer specific for the region of interest, wherein the second primer comprises an affinity moiety,
amplifying the region of interest, thereby producing a double stranded polynucleotide amplification product comprising the single stranded target polynucleotide molecular size standard comprising the label and the mobility modifier, and a complementary affinity moiety strand,
contacting the reaction mixture with a binding moiety specific for the affinity moiety,
binding the double stranded polynucleotide to the binding moiety, removing the unbound unincorporated reaction components, and releasing the labeled single stranded target polynucleotide molecular size standard.

17. The method according to claim 16 further comprising a plurality of regions of interest and a plurality of primer pairs, wherein a plurality of labeled single stranded target polynucleotide molecular size standards is formed.

18. A method for isolating a labeled single stranded target polynucleotide comprising, forming a PCR mixture comprising,
a. a polynucleotide region of interest,
b. a first primer specific for the region of interest,
c. a second primer specific for the region of interest, wherein the second primer comprises an affinity moiety,
amplifying the region of interest, whereby a double stranded polynucleotide amplification product is produced, comprising an unlabelled single stranded target polynucleotide complement, and a complementary affinity moiety strand,
contacting the reaction mixture with a binding moiety specific for the affinity moiety,
binding the double stranded polynucleotide amplification product to the binding moiety,
removing the unbound unincorporated reaction components, and eluting and removing the unlabelled singled stranded target polynucleotide,
providing,
a. a polymerase,
b. a primer complementary to the bound second strand, wherein the primer further comprises a mobility modifier, and,
c. at least one dye-labelled nucleotide,
performing an extension reaction to form a labeled single stranded target polynucleotide, and,
releasing the labeled single stranded target polynucleotide.

19. The method according to claim 18 wherein the labeled single stranded polynucleotide is analyzed by a mobility-dependent analysis technique.

20. The method according to claim 19 wherein the mobility-dependent analysis technique is capillary electrophoresis.
